(19) United States
(12) Patent Application Publication
(10) Pub. No.: US 2015/0238350 A1
(43) Pub. Date: Aug. 27, 2015

(54) TEMPERATURE CONTROLLER FOR CONTROLLING BODY TEMPERATURE

(71) Applicant: KONINKLIJKE PHILIPS N.V., EINDHOVEN (NL)

(72) Inventors: Tim Johannes Willem Tijs, Stemproy (NL); Paul Marcel Carl Lemmens, Veghel (NL); Tim Elisabeth Joseph Weysen, Maastricht (NL); Gijs Antonius Franciscus Van Elswijk, Eindhoven (NL)

(73) Assignee: KONINKLIJKE PHILIPS N.V., EINDHOVEN (NL)

(21) Appl. No.: 14/413,428

(22) PCT Filed: Jun. 28, 2013
(86) PCT No.: PCT/IB2013/055322

§ 371 Date: Jan. 8, 2015

Related U.S. Application Data
(60) Provisional application No. 61/671,169, filed on Jul. 13, 2012.

(51) Int. Cl. A61F 7/00

(52) U.S. Cl. CPC A61F 7/0085; A61F 2007/0054; A61F 2007/0056; A61F 2007/0095; A61F 2007/0094

(57) ABSTRACT

The invention relates to a method for avoiding that brown adipose tissue is stimulated due to low body temperatures. By avoiding such stimulation of the brown adipose tissue, unintended uptake of a radioactive glucose tracer in the brown adipose tissue may be minimized which may be important in PET imaging to avoid generation of false positive images. An embodiment of the invention suggests an apparatus (100) configured with a thermoregulation device (101) for heating e.g. the chest area and a controller (103) for controlling the thermoregulation device on the basis of a temperature reference (Tr) and measurements of the body temperature, e.g. a local body temperature (Tbm).

### FIELD OF THE INVENTION

The invention relates generally to the field of medical devices, specifically to a controller for controlling body temperature.

### BACKGROUND OF THE INVENTION

Before a patient is scanned in a Position Emission Tomography (PET) scanner the patient may receive a radioactive glucose tracer such as FDG. After having received the tracer, the patient needs to rest so that the tracer can be taken up by the body. The tracer is primarily taken up in parts of the body which are highly hypermetabolic. Tumors are hypermetabolic and, therefore, appear as distinctive, e.g. highlighted areas in images from the PET scanner.

However, other parts of the body may also take up the tracer and, therefore, generate false positive results since such other parts may also be visible as distinctive areas in the image. For example, it is known that brown adipose tissue in a stimulated state may take up glucose tracers.

Accordingly, there is a need for developing methods which are able to generate improved PET images and which are able to minimize the possibility for generating false positive results.

US2012055187 discloses a garment which comprises (a) a first portion constructed from stretchable material that is form fitting to a user's physique and that extends across any of a user's collar bone, neck, upper spine, or armpit area; and (b) a cooling composition disposed in said first portion, said cooling composition comprising a material that applies a cooling effect to the collar bone area of the user.

The garment disclosed in US2012055187 contains passive cooling packs intended for cooling the body in order to obtain thermal stimulation of brown adipose tissues in the body of the user and, therefore, does not provide a satisfactory solution for the current need.

Accordingly, the inventor of the present invention has appreciated that improvements within medical devices for use in the process of obtaining medical images is of benefit, and has in consequence devised the present invention.

### SUMMARY OF THE INVENTION

It would be advantageous to achieve improvements within medical devices used for obtaining medical images, particularly PET generated images. It would also be desirable to make PET generated images more reliable. In general, the invention preferably seeks to mitigate, alleviate or eliminate problems in medical imaging such as PET imaging caused by unintended uptake of glucose tracers in parts of the body such as in brown adipose tissue since such uptake may lead to false positive clinical results. Furthermore, it may be seen as an object of the present invention to provide a method that solves other problems of the prior art.

To better address one or more of these concerns, in a first aspect of the invention an apparatus for controlling body temperature Tb of a patient is presented that comprises:
a thermoregulation device configured for heating at least a part of the body of the patient, wherein the heating is dependent upon a control input,
a temperature sensor configured for measuring a body temperature of at least a part of the body,
a control system configured for determining the control input on the basis of a reference temperature and the measured body temperature.

By heating a part of the body, for example a part such as the upper chest region and/or the neck which may contain brown adipose tissue, the thermogenesis function of the brown adipose tissue may not be stimulated or may be less stimulated as compared to a situation wherein the patient rests in a normally heated room. Therefore, due to the heating, a radioactive glucose tracer may not be taken up or may be taken up to a less degree by the heated part of the body. Consequently, generation of false positive results may be avoided or limited since only little radioactivity is present in the heated brown adipose tissue when the thermogenesis function is not activated or not fully activated.

By controlling the heating in response to the measured body temperature, e.g. a measured local body temperature measured e.g. at the chest region, it may be possible to improve the heating process so that the patient feels as little discomfort as possible. For example, it may be possible to heat the body part so that overheating of the body part is avoided, i.e. so that the body is not heated to a temperature exceeding the reference temperature.

Similarly, by the controlled heating it may be possible to stabilize the body temperature at the reference temperature which preferably is within a “thermo-neutral range”. In this “thermo-neutral range” body warming is effective and without thermoregulatory counteractions of the body and without major loss of patient comfort. The thermo-neutral range may be the range from 18 to 30 degrees Celsius. Since most PET facilities follow very strict time schedules and large numbers of patients are scheduled every day—e.g. because scanner time is expensive, because the cost of FDG is fairly high, or because the FDG can only be used at a pre-scheduled time because of radioactive decay—the time to prepare a patient for the FDG injection should be minimized. Such time minimization may be achievable by the controlled heating since the controller may enable fast heating without causing discomfort.

Furthermore, the gentle heating provided by the temperature control apparatus may have a relaxing effect on the patient which may be nervous about the possible serious results from the PET scanner.

In an embodiment the temperature control apparatus further comprises a temperature scheduler for determining the reference temperature from temperature schedule data. The temperature schedule data could prescribe a schedule, possibly a time dependent schedule, of temperatures which should be used as reference temperatures for the heating process. The temperature schedule data may advantageously prescribe body warning temperatures which are suited for a patient with given patient characteristics, e.g. weight.

The temperature schedule data may contain a single temperature value such as the reference temperature Tr or a plurality of temperature values or temperature related values such as rate-of-change values describing how fast the measured body temperature is allowed to change.

In an embodiment the temperature control apparatus is configured to receive the temperature schedule data from a storage. For example, a plurality of data records containing temperature schedule data may be stored in a storage which may be an associated external storage or a storage comprised by the temperature control apparatus. The data records may contain patient characteristics associated with the temperature schedule data so that a set of temperature schedule data suitable for a patient with given patient characteristics can be retrieved from the storage by comparing the stored patient characteristics with the patient characteristics of the present patient.

The temperature control apparatus may be configured to retrieve the temperature schedule data from the database or storage on the basis of input data to the database containing patient characteristics of the patient.

In an embodiment the temperature control apparatus comprises a user input device wherein the user input device is configured to determine the temperature schedule data on the basis of input data to the user input device containing patient characteristics of the patient and/or temperature values relating to a desired temperature schedule. The user input device may determine the temperature schedule data by comparing the patient characteristics with patient characteristics stored in a storage (e.g. a storage in the user input device) having associated temperature schedule date and retrieving the temperature schedule data which provided the best match with the current patient characteristics. Alternatively, the temperature schedule data may be determined directly on the basis of temperature values inputted by a user. For example, the user may input the desired end temperature from which other intermediate temperatures and a start temperature may be determined, by the user input device.

In an embodiment the temperature control apparatus comprises an input enabling the patient to input patient information relating to how the heat from the thermoregulation device is experienced. The patient'information may be used to modify e.g. the reference temperature if the patient provided input information indicating that the temperature is too high. Accordingly, the temperature control apparatus may be configured to change the reference temperature on the basis of the patient information.

Similarly the temperature control apparatus may be configured to change the reference temperature on this basis of an outdoor temperature. A patient which enters a hospital may be exposed to low outdoor temperatures. Low outdoor temperatures may stimulate brown adipose tissue and, therefore, the reference temperature may advantageously be modified, e.g. increased, in order to avoid brown adipose stimulation due to the outdoor temperature.

The temperature control apparatus may comprise an alarm configured to generate an alarm on the basis of the value of the measure body temperature. Accordingly, clinical personnel may be notified, e.g. if a patient is not warmed up as desired.

The thermoregulation device may be configured to apply the heat to the patient by heating the skin. Thus, the thermoregulation device may heat the skin directly, e.g. via contact with the skin or the clothes worn by the patient, or the thermoregulation device may heat the skin indirectly e.g. via infra-red radiation.

In an embodiment the thermoregulation device comprises a reservoir for containing a liquid, wherein the temperature of the liquid can be controlled on the basis of the control input.

In an embodiment the thermoregulation device is configured for heating and cooling at least a part of the human body. By enabling the thermoregulation device to also cool the body improved temperature control may be achieved.

A second aspect of the invention relates to a system for controlling the body temperature, the system comprises:
the apparatus according to the first aspect, and
a database configured to contain:
   patient characteristics of patients which have received a heating treatment from a thermoregulation device, and
   temperature information of the heating treatment, wherein the temperature information comprises a reference temperature and/or a measured body temperature, wherein the apparatus is configured to receive the temperature information from the database based on patient characteristics of the patient.

The database may further be configured to contain information relating to the effect of the heating treatment, wherein the information comprises one or more of:
a) measured temperature values of body temperature,
b) comfort information relating to how the heat from the thermoregulation device is experienced, and
c) scanning results showing a degree of uptake of a glucose based tracer given to the patient in a body part exposed to the heating treatment.

A third aspect of the invention relates to a method for controlling body temperature of a patient, comprising:
heating at least a part of the body, wherein the heating is generated on the basis of a control input by use of a thermoregulation device being responsive to the control input,
measuring a body temperature of at least part of the body,
determining the control input on the basis of a reference temperature and the measured body temperature by use of a control system.

In summary the invention relates to a method for avoiding that brown adipose tissue is stimulated due to the low body temperatures. By avoiding such stimulation of the brown adipose tissue, unintended uptake of a radioactive glucose tracer in the brown adipose tissue may be minimized which may be important in PET imaging. An embodiment of the invention suggests an apparatus configured with a thermoregulation device for heating e.g. the chest area and a controller for controlling the thermoregulation device depending upon a temperature reference and measurements of the body temperature, e.g. a local body temperature.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which

FIG. 1 schematically illustrates an apparatus 100 and a system 199 for controlling body temperature,

FIG. 2 illustrates temperature schedule data 201-206 applicable to the temperature control apparatus 100, and

FIG. 3 illustrates an example of a thermoregulation device 101 for generating localized heat where the thermoregulation device 101 is integrated in a bedside unit 300.

### DESCRIPTION OF EMBODIMENTS

Before a patient is scanned in a Position Emission Tomography (PET) scanner or other scanner with similar properties the patient receives a radioactive glucose tracer, e.g. via an injection. The radioactive glucose tracer (FDG) may be the 2-[¹⁸F]-2-deoxy-D-glucose tracer. After having received the tracer, the patient needs to rest, e.g. for an hour, so that the tracer can be taken up by the body. The tracer is primarily taken up in parts of the body which are highly hypermetabolic, i.e. which are “hungry for sugar”. Tumors are highly hypermetabolic and, therefore, appears as distinctive areas in images from the PET scanner. However, parts of the body containing brown adipose tissue (BAT) also has hypermetabolic capabilities and, therefore, PET images will show such BAT body parts as false positive areas in the image. Accordingly, uptake of the tracer in body parts containing BAT may hinder correct medical diagnosis.

It is known that the glucose tracer is primarily taken up in BAT regions when the body or a part of the body has relatively low temperatures. This is due to the thermogenesis function of the brown adipose tissue which helps to heat the body.

Studies indicate that uptake of the glucose tracer in BAT regions can be minimized by heating the body or parts of the body before the glucose tracer is given to the patient. This may be achieved for example by use of blankets or by ensuring a high room temperature. The following embodiments provide alternative solutions for controlling the temperature of the patient's body or a part of the patient's body.

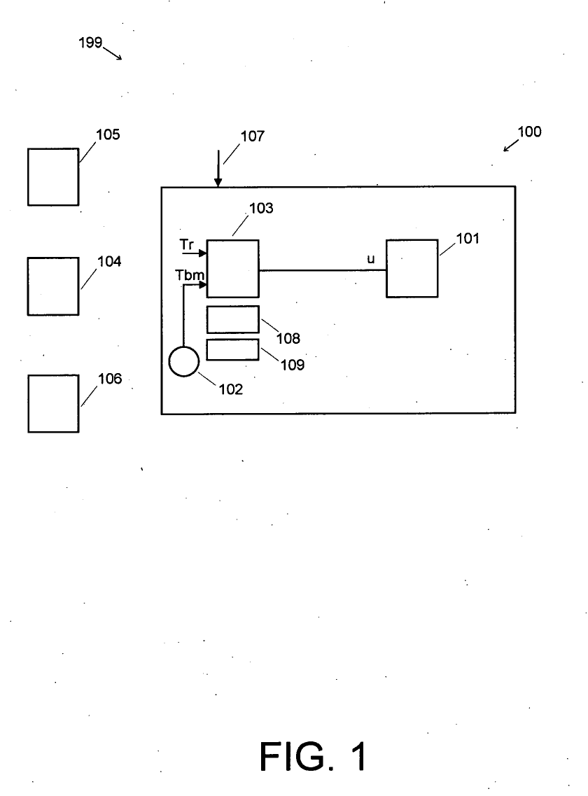

FIG. 1 shows an embodiment of an apparatus 100 for controlling body temperature (Tb) of a patient. The temperature control apparatus 100 comprises a thermoregulation device 101 for heating at least a part of the human body.

The thermoregulation device 101 may be an infra-red heater, a heater containing electrical heating pads, a water-based heater or other heater configured to apply heat to the patient by heating the skin. Experience has shown that a water based heater has better capabilities for stabilizing body temperature around a desired body temperature than several other heaters. However, where heating of a body part is required over a distance infra-red heating may be preferred.

The thermoregulation device 101 is controllable so that heat from the heater can be generated depending upon a control input u. The heater may be a simple heater which is controllable either to generate a preset or fixed heating or no heating depending upon the control input u. Preferably, the heater is controllable to generate different levels of heating, e.g. different temperature values, on the basis of the control input u. For example, different levels of heating from a water based heater may be achieved by increasing water temperature.

The water/liquid based heater or thermoregulation device may have a closed or open water/liquid reservoir wherein the temperature of water or other liquid can be controlled dependent upon the control input u. For example, water can be heated by an electrical heater such as a heating wire located in the reservoir. Alternatively, the water based heater may be configured to circulate water so the water heated by an electrical heater is directed into a reservoir configured to heat the patient and so that water from the reservoir is directed back the electrical heater.

The thermoregulation device 101, such as the liquid based thermoregulation device, may be configured to heat and/or cool certain parts of the body. Since brown adipose tissue is primarily located at the neck and chest area of the human body, the thermoregulation device may be configured as a wearable device or garment. Alternatively, the thermoregulation device may be integrated in a chair, e.g. in the neck support area of the chair, or in other furniture such as a bed.

Herein a patient may refer to a person who receives the heating treatment, or persons who have previously received the heating treatment. Clinical staff refers to e.g. doctors, nurses or other people which may operate the heating apparatus 100. A user may be a patient or a person of the clinical staff.

In an embodiment the thermoregulation device 101 is configured for heating and cooling at least a part of the human body. The heating and cooling thermoregulation device 101 may be a simple device which is controllable to generate a single level of heating, a single level of cooling or no heating/cooling depending upon the control input u. Alternatively, the thermo regulation device is controllable to generate different levels of heating and cooling, i.e. different temperature values, depending upon the control input u.

The temperature control apparatus 100 further comprises a temperature sensor 102 configured for measuring the body temperature Tb of the patient. The temperature sensor 102 may be configured to measure an average body temperature of the entire patient body, or the temperature sensor may be configured to measure a body temperature of a part of the patient body such as an average temperature of the chest region. For example, the temperature sensor 102 may be an infra-red sensor configured to measure the infra-red electro-magnetic radiation emitted by a part of the patient body or the entire body.

The apparatus 100 also comprises a control system 103 configured for determining the control input u on the basis of a reference temperature Tr and the measured body temperature Tbm. For example, the control system 103 may comprise a look-up table or a mathematical function configured to determine the control input u depending upon various combinations of reference temperatures Tr and measured body temperatures Tbm.

The thermoregulation device 101 having only heating capabilities may be responsive to the control input u, e.g. so that if u is greater than zero the thermoregulation device will generate heat and if u is equal to zero then the heater will not generate heat. Possibly, the temperature of the generated heat may correspond to the value of the control input u, e.g. by a linear or non-linear relationship. The thermoregulation device having both heating and cooling capabilities may be responsive to the control input u in a similar way, e.g. so that if u is less than zero the thermoregulation device will generate cooling, where the temperature of the cooling may or may not be adjustable depending upon the value of the control input u.

The control system 103 may be configured as a feedback system comprising an input for receiving the reference temperature Tr, an input for receiving the measured body temperature Tbm, a calculation unit for determining a temperature difference ΔT between the reference temperature and the measured body temperature, and a control law for determining the control input u depending upon the temperature difference ΔT. The control law could include the control law of a proportional (P) controller, a proportional-integral (PI) controller or other suitable control law.

The reference temperature Tr may be set to the desired body temperature which should lower the uptake of the radioactive glucose tracer in brown adipose tissue. Thus, by providing a reference temperature it is possible to increase or decrease the body temperature of the body or part of the body of a patient by use of the temperature apparatus 100 so that the body temperature approximates the desired reference temperature.

The apparatus 100 may be configured with a temperature scheduler 104 for determining the reference temperature Tr from temperature schedule data.

The temperature control apparatus 100 may be comprised by a system 199 which additionally comprises a database 105 configured to contain patient characteristics from previous patients and temperature information of the heating treatment wherein the apparatus 100 is configured to receive the temperature information from the database based on patient characteristics of the current patient.

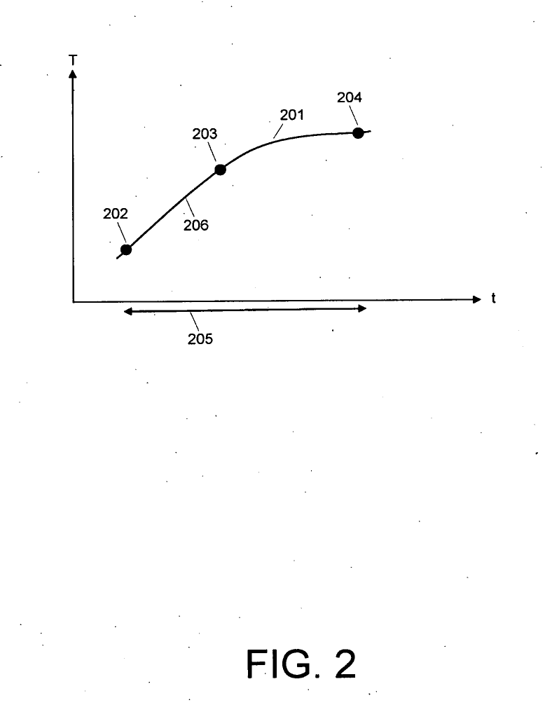

FIG. 2 shows an example of temperature schedule data. The temperature schedule data could contain temperatures T which indicate a suitable start temperature 202, a desired end temperature 204 and/or suitable intermediate temperatures 203. The temperature schedule data may also contain time values 205 suitable for indicating a time period wherein the body temperature Tb should be controlled or a time period wherein the body temperature Tb should be changed by a given temperature increase. The temperature schedule data could also contain rate-of-change values 206 indicating e.g. a maximum allowable rate of change of the body temperature Tb or reference temperature Tr.

The temperature schedule data could also be in the form of a temperature curve 201 defining suitable body temperatures Tb for a heating process of a body part of a patient.

The values of the temperature schedule data 201-206 and, therefore, also the reference temperature Tr may be within a range from 18 to 30 degrees Celsius.

The temperature schedule data 201-206 may be given as a function of time or independent of time.

Thus, the temperature scheduler 102 is able to determine reference temperatures for the control system 103 so that the body temperature Tb can be controlled according to temperature schedule data 201-206, e.g. so that the rate-of-change of the body temperature Tb can be controlled. For example, it may be desired that a patient's body temperature of a part of the body increases by a given amount (e.g. 2 degrees Celsius) within a given time (e.g. 20 minutes).

The apparatus 100 may receive the temperature schedule data from a storage 105 such as an external storage, e.g. database, connectable with the apparatus 100 or integrated with the apparatus 100.

The data stored in the storage or database 105 may be arranged in data records associated with different previous patients.

The apparatus may be configured to retrieve the temperature schedule data from the storage or database on the basis of input data to the database containing patient characteristics of the patient.

The temperature schedule data 201-206 may be generated from patient characteristics of patients which previously have received a heating treatment. For example, patient characteristics such as weight may be useful for selection of suitable temperature schedule data. For example, a relatively heavy patient may require higher heating temperatures and/or a longer heating period.

A user input device 106 connectable with the apparatus 100 or integrated with the apparatus 100 as shown in FIG. 1 may be configured to determine the temperature schedule data on the basis of input data such as patient characteristics. Alternatively or additionally, the user input device 106 may be configured to receive temperature values relating to a desired temperature schedule. For example, if no patient characteristics are available for a patient with certain patient characteristics a suitable final temperature 204 (i.e. a reference temperature Tr) and possibly start and intermediate temperatures may be inputted to the input device 106. The input data may be provided to the user input device 106 via any suitable user interface, such as a touch screen.

In order to be able to find suitable temperature schedule data from previous heating treatments the storage or database 105 may contain patient characteristics (age, weight, etc.)

of patients which have received a heating treatment and temperature information of the heating treatment. The temperature information may be values of temperature schedule data used for the heating treatment for the patient. The storage or database 105 may further contain information relating to the effect of the heating treatment, for example measured temperature values of body temperature Tbm, comfort information indicating how the heat from the thermoregulation device is experienced, and or scanning results showing a degree of uptake of the glucose based tracer given to the patient in a body part exposed to the heating treatment.

The temperature control apparatus 100 may be configured to receive the temperature information from the database based on patient characteristics of the current patient. The patient characteristics may be inputted to the apparatus 100 via a user interface. Based on the patient characteristics the apparatus 100 may send a request to the database to find temperature information having associated patient characteristics which corresponds to or is close to the patient's characteristics.

The temperature information may further be selected on the basis of one or more of: a) the measured temperature values of body temperature, b) comfort information relating to how the heat from the thermoregulation device is experienced, and c) scanning results showing a degree of uptake of a glucose based tracer given to the patient in a body part exposed to the heating treatment. The selection of temperature information may be performed by the database or the apparatus 100.

The apparatus 100 may be configured with an input 107 enabling the patient to input patient information relating to how the heat or temperature from the thermoregulation device is experienced. For example, if the patient feels that the temperature of the heat is uncomfortable the patient may give an input to the apparatus via the input 107 indicating the uncomfortable experience. The input 107 may be any suitable user interface and may be integrated with the user input device 106.

The apparatus 100 may be configured to change the reference temperature or temperature values of the temperature schedule data 201-206 depending upon the patient information, e.g. so that if the patient provides an input to the apparatus 100 for indicating that the patient feels that the heating is uncomfortable, the apparatus may lower the reference temperature or temperature values of the temperature schedule data.

Similarly, the apparatus may be configured with an input enabling the patient to input information indicating that the temperature of the heat from the thermoregulation device may be increased without causing discomfort and the apparatus 100 may be configured to increase the reference temperature, temperature values of the temperature schedule data or the rate-of-change of the temperature schedule data on the basis of this input.

If outdoor temperatures are low the human body may react by activating the thermogenesis function of brown adipose tissue regions to warm the body. It may be possible to deactivate such activated brown adipose tissue by heating regions of the body by use of the temperature control apparatus 100. Accordingly, the apparatus 100 may be configured to change the reference temperature or the temperature schedule data depending upon an outdoor temperature, e.g. so that if outdoor temperatures are below e.g. zero degrees Celsius then the reference temperature or temperature schedule values are modified, e.g. increased.

The apparatus may be configured with an alarm 108 which is configured to generate an alarm depending upon the value of the measured body temperature Tbm. Thus, if the measured body temperature Tbm does not increase to the desired body temperature Tb during the heating process the staff may be notified by an alarm.

In order to update the storage or database 105 with new data relating to a heating process which can be used later on for selection of temperature schedule data suitable for a new patient with certain patient characteristics the apparatus 100 may be configured with an analysis function 109 for generating accessible output data containing information relating to the applied temperature schedule data, reference temperatures and the measured body temperature Tbm. Advantageously, the analysis function 109 may also generate output data relating to the patient characteristics together with the other output data. Alternatively or additionally, the output data may contain evaluation data from an evaluation of the performed heating treatment which could indicate if the heating process has been successful or not. For example, the evaluation data may indicate if the brown adipose tissue regions of a patient showed “false positive” results in the PET image.

Further relevant output data which may be generated by the analysis function 109 comprises comfort information relating to how the heat from the thermoregulation device is experienced, and scanning results showing a degree of uptake of the glucose tracer given to the patient in a body part exposed to the heating treatment.

The output data from the analysis function 109 may be stored in the storage or database so that reference temperature values or temperature schedule data which are suitable for a new patient can be retrieved from the storage or database, e.g. by searching for data entries containing data indicating a successful heating treatment and/or patient characteristics which correspond to the new patient's characteristics.

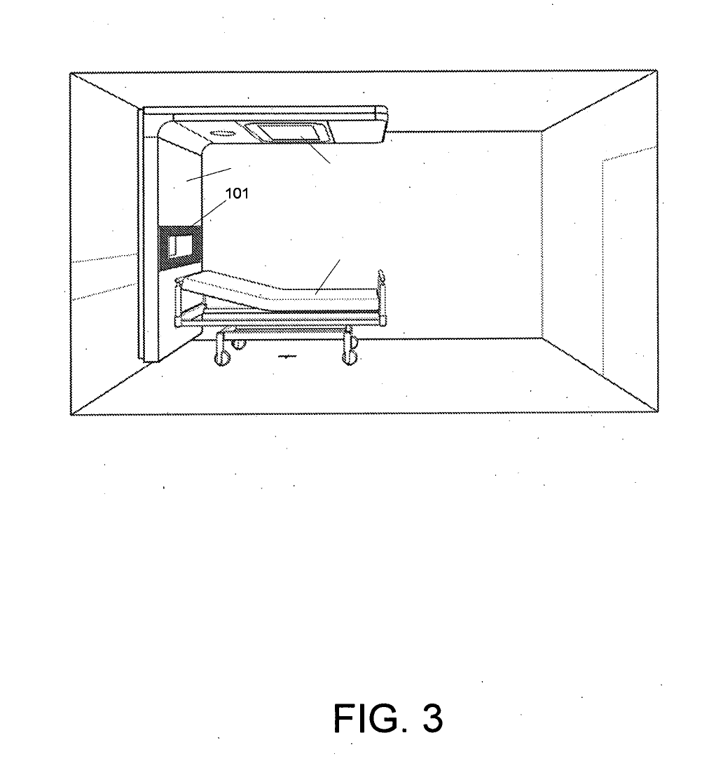

FIG. 3 shows a bedside unit 300 containing e.g. a display 301 for showing video for a patient located in the bed 302. The temperature control apparatus 100 is integrated in the bedside unit 300. In FIG. 3 the thermoregulation device 101 is configured as an infrared LED array positioned close to the head-end of the bed and, thereby, configured to apply heat to the upper body area of a patient located in the bed: The temperature sensor 102 is a camera (not shown) which is also integrated in the bedside unit 300.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. An apparatus for controlling body temperature of a patient, the apparatus comprises:
a thermoregulation device configured for heating at least a part of the body of the patient, wherein the heating is dependent upon a control input (u),
a temperature sensor configured for measuring a body temperature (Tbm) of at least a part of the body,
a control system configured for determining the control input (u) on the basis of a reference temperature (Tr) and the measured body temperature (Tbm), and
an input enabling the patient to input patient information relating to how the heat from the thermoregulation device is experienced.

2. An apparatus according to claim 1, further comprising a temperature scheduler for determining the reference temperature (Tr) from temperature schedule data.

3. An apparatus according to claim 2, wherein the apparatus is configured to receive the temperature schedule data from a storage.

4. An apparatus according to claim 3, wherein the storage is a database and where the apparatus is configured to retrieve the temperature schedule data from the database on the basis of input data to the database containing patient characteristics of the patient.

5. An apparatus according to claim 2, further comprising a user input device, wherein the user input device is configured to determine the temperature schedule data on the basis of input data to the user input device containing patient characteristics of the patient and/or temperature values relating to a desired temperature schedule.

6. (canceled)

7. An apparatus according to claim 1, wherein the apparatus is configured to change the reference temperature (Tr) on the basis of the patient information.

8. An apparatus according to claim 1, wherein the apparatus is configured to change the reference temperature (Tr) on the basis of an outdoor temperature.

9. An apparatus according to claim 1, further comprising an alarm configured to generate an alarm on the basis of the value of the measure body temperature (Tbm).

10. An apparatus according to claim 1, wherein the thermoregulation device is configured to apply the heat to the patient by heating the skin.

11. An apparatus according to claim 1, wherein the thermoregulation device comprises a reservoir for containing a liquid, wherein the temperature of the liquid can be controlled on the basis of the control input (u).

12. An apparatus according to claim 1, wherein the thermoregulation device is configured for heating and cooling at least a part of the human body.

13. A system for controlling body temperature of a patient, the system comprises:
the apparatus according to claim 1, and
a database configured to contain:
patient characteristics of patients which have received a heating treatment from a thermoregulation device, and
temperature information of the heating treatment, wherein the temperature information comprises a reference temperature (Tr) and/or a measured body temperature (Tbm),
wherein the apparatus is configured to receive the temperature information from the database based on patient characteristics of the patient.

14. A system according to claim 13, wherein the database is further configured to contain information relating to the effect of the heating treatment, wherein the information comprises one or more of:
a) measured temperature values of body temperature (Tbm),
b) comfort information relating to how the heat from the thermoregulation device is experienced, and
c) scanning results showing a degree of uptake of a glucose based tracer given to the patient in a body part exposed to the heating treatment.

15. A method for controlling body temperature of a patient, comprising:
heating at least a part of the human body, wherein the heating is generated on the basis of a control input (u) by use of a thermoregulation device being responsive to the control input (a),
measuring a body temperature (Tbm) of at least a part of the body,
determining the control input (u) on the basis of a reference temperature (Tr) and the measured body temperature (Tbm) by use of a control system, enabling the patient to input patient information relating to how the heat of the thermoregulation device is experienced.
